SEQUENCE LISTING (22 sequences)

SEQ ID NO: 1 - protein, 16 aa, unknown
  Salmonella species/serovar
VASTASDEARESSRKS

SEQ ID NO: 2 - protein, 16 aa, unknown
  Salmonella species/serovar
NNHSVENSSQTASQSV

SEQ ID NO: 3 - protein, 15 aa, unknown
  Salmonella species/serovar
GQYAATQERSEQQIS

SEQ ID NO: 4 - protein, 14 aa, unknown
  Salmonella species/serovar
LGIKDSNKQISPEH

SEQ ID NO: 5 - protein, 15 aa, unknown
  Salmonella species/serovar
LNMKKTGTDATKNLN

SEQ ID NO: 6 - protein, 409 aa, unknown
  Salmonella species/serovar
MLISNVGINPAAYLNNHSVENSSQTASQSVSAKDILNSIGISSSKVSDLGLSPTLSAPAPGVLTQTPGTITSFLKASIQNTDMNQDLNALANNVTTKANEVVQTQLREQQAEVGKFFDISGMSSSAVALLAAANTLMLTLNQADSKLSGKLSLVSFDAAKTTASSMMREGMNALSGSISQSALQLGITGVGAKLEYKGLQNERGALKHNAAKIDKLTTESHSIKNVLNGQNSVKLGAEGVDSLKSLNMKKTGTDATKNLNDATLKSNAGTSATESLGIKDSNKQISPEHQAILSKRLESVESDIRLEQNTMDMTRIDARKMQMTGDLIMKNSVTVGGIAGASGQYAATQERSEQQISQVNNRVASTASDEARESSRKSTSLIQEMLKTMESINQSKASALAAIAGNIRA

SEQ ID NO: 7 - DNA, 1230 bp, unknown
  Salmonella species/serovar
atgttaattagtaatgtgggaataaatcccgccgcttatttaaataatcattctgttgagaatagttcacagacagcttcgcaatccgttagcgctaaagatattctgaatagtattggtattagcagcagtaaagtcagtgacctggggttgagtcctacactgagcgcgcctgcgccaggggtattaacgcaaacccccggaacgatcacgtcctttttaaaagccagtattcaaaataccgacatgaatcaggatttgaatgctctggcaaataatgtcacgactaaagcgaatgaggttgtgcaaacccagttacgcgagcagcaggcagaagtcggaaagttttttgatattagcggaatgtcttccagtgccgttgcgctgttggctgccgcgaatacgttaatgctgacgttgaaccaggctgatagcaaactgtctggtaagttgtcattagtcagttttgatgcagctaaaacgacggcaagctccatgatgcgcgaagggatgaatgcgttgtccggtagtatttcccagagcgcgcttcagttggggatcactggcgtgggcgccaaactggaatataaggggctgcagaatgaaagaggcgcgcttaaacataatgccgcgaagatcgataaactgaccactgaaagccacagtattaaaaacgtgctgaacgggcagaatagcgtcaaactcggtgctgaaggcgtcgattctctgaaatcgttaaatatgaagaaaaccggtaccgatgcgacgaaaaatcttaatgatgcgacgcttaaatctaatgccggaaccagcgccacggaaagtctgggtattaaagacagtaataaacaaatctcccctgaacatcaggctattctgtcgaaacgtcttgagtctgtcgaatccgatattcgtcttgagcagaataccatggatatgacccgaatcgatgcgcgcaagatgcagatgacgggcgatctgattatgaagaactcggtcacggtcggtggtattgcaggggcgtccgggcagtacgccgctactcaggaacgttccgagcagcaaattagccaggtgaataaccgggttgccagcaccgcatcggacgaagcccgtgaaagttcacgtaaatcgaccagcctgattcaggaaatgctgaaaacaatggagagcattaaccagtcgaaagcatccgcactcgctgctatcgcaggcaatattcgcgcttaa

SEQ ID NO: 8 - DNA, 30 bp, unknown
  Salmonella species/serovar
ggattgcgaagctgtctgtgaactattctc

SEQ ID NO: 9 - DNA, 15 bp, unknown
  Salmonella species/serovar
cgtcgcatcggtacc

SEQ ID NO: 10 - DNA, 18 bp, unknown
  Salmonella species/serovar
gagatttgtttattactg

SEQ ID NO: 11 - DNA, 20 bp, unknown
  Salmonella species/serovar
gaacgttcctgagtagcggc

SEQ ID NO: 12 - DNA, 28 bp, unknown
  Salmonella species/serovar
cacgggcttcgtccgatgcggtgctggc

SEQ ID NO: 13 - DNA, 30 bp, unknown
  Salmonella species/serovar
atcccgccgcttatttaaataatcattctg

SEQ ID NO: 14 - DNA, 18 bp, unknown
  Salmonella species/serovar
cgatagcagcgagtgcgg

SEQ ID NO: 15 - DNA, 30 bp, unknown; identical to SEQ ID NO: 13
  Salmonella species/serovar

SEQ ID NO: 16 - DNA, 22 bp, unknown
  Salmonella species/serovar
gagtgcggatgctttcgactgg

SEQ ID NO: 17 - DNA, 25 bp, unknown
  Salmonella species/serovar
tagttcacagacagcttcgcaatcc

SEQ ID NO: 18 - DNA, 30 bp, unknown
  Salmonella species/serovar
ttaatgctctccattgttttcagcatttcc

SEQ ID NO: 19 - DNA, 24 bp, unknown
  Salmonella species/serovar
acagacagcttcgcaatccgttag

SEQ ID NO: 20 - DNA, 29 bp, unknown
  Salmonella species/serovar
atgctctccattgttttcagcatttcctg

SEQ ID NO: 21 - DNA, 24 bp, unknown
  Salmonella species/serovar
acagacagcttcgcaatccgttag

SEQ ID NO: 22 - DNA, 22 bp, unknown
  Salmonella species/serovar
tattgcctgcgatagcagcgag

(19) United States
(12) Patent Application Publication
(10) Pub. No.: US 2013/0115639 A1
(43) Pub. Date: May 9, 2013

(54) METHOD FOR DETECTING A SALMONELLA INFECTION

(71) Applicants: Udo Meyer, Hastorf (DE); Alain Steinmann, Rostock (DE); Heike Fahlandt, Niendorf (DE); Babette Schulz, Rostock (DE)

(72) Inventors: Udo Meyer, Hastorf (DE); Alain Steinmann, Rostock (DE); Heike Fahlandt, Niendorf (DE); Babette Schulz, Rostock (DE)

(73) Assignee: BIOSERV ANALYTIK UND MEDIZINPRODUKTE GMBH, Rostock (DE)

(21) Appl. No.: 13/642,629

(22) PCT Filed: Apr. 14, 2011
(86) PCT No.: PCT/DE2011/000460

§ 371 Date: Jan. 14, 2013

(30) Foreign Application Priority Data
Apr. 23, 2010 (DE) 10 2010 018 085.8

(51) Int. Cl. G01N 33/569

(52) U.S. Cl. 435/7.94; 530/387.9

(57) ABSTRACT

The invention relates to a diagnostic method to detect a salmonellae infection and/or salmonellae contamination. Fields of use include medicine, veterinary medicine and various branches of industry.

The goal of the invention is to develop a reliable test to identify salmonellae. It is based on the task of designing a method that allows the earliest possible identification and covers all important serovars.

The diagnostic method to detect a salmonellae infection/contamination in accordance with the invention is characterised in that the SipC of all known salmonellae serovars excreted by the salmonellae can be identified in excretions of patients/animals, carcasses, eggs and food/animal feed. This identification is carried out by means of immunochemical systems using mono- and polyclonal antibodies that are directed against the following peptide sequences:

(SEQ ID NO 1)
NH₂-V-A-S-T-A-S-D-E-A-R-E-S-S-R-K-S-COOH,
(SEQ ID NO 2)
NH₂-N-N-H-S-V-E-N-S-S-Q-T-A-S-Q-S-V-COOH,
(SEQ ID NO 3)
NH₂-G-Q-Y-A-A-T-Q-E-R-S-E-Q-Q-I-S-COOH,
(SEQ ID NO 4)
NH₂-L-G-I-K-D-S-N-K-Q-I-S-P-E-H-COOH
and
(SEQ ID NO 5)
NH₂-L-N-M-K-K-T-G-T-D-A-T-K-N-L-N-COOH.

The invention relates to a diagnostic method to detect a salmonella infection and/or salmonella contamination. Fields of use include medicine, veterinary medicine and various branches of industry.

Salmonellae are motile, Gram-negative, rod-shaped bacteria. Taxonomically speaking, a differentiation is made between salmonellae by the occurrence of somatic (O) and flagellar (H) antigens and they are classified into serovars in a classification system and characterised by means of their sero-formula. Around 2,400 salmonella serovars have been described to date. However, only 20-30 serovars are significant as pathogens of epidemiological illnesses in practice (e.g. S. typhi, S. paratyphi A, B, C as well as a large number of enteritis pathogens).

Salmonelloses in humans are mainly triggered by the intake of infected or contaminated food. Transmission from animals to humans through contact is of little significance. Direct or indirect transmission from human to human is also a rare event, though this can occur as a hospital infection in predisposed persons or in hygienically unfavourable circumstances. The main primary sources of infections are food from poultry, pigs and cattle. Since the animals are rarely affected themselves, the identification of the pathogen and/or antibody is very important in both veterinary medicine and the food industry.

The infective dose for healthy adults is 10⁴ to 10⁶ germs. The incubation period is between 5 and 72 hours depending on the infective dose. An enteritis-salmonellae-infection manifests itself through diarrhea, nausea, vomiting and a light to moderate fever. The symptoms usually only last between a few hours and a day, though they can lead to the death of old and weak persons or children.

The germs are normally excreted for an average of 3 to 6 weeks after a salmonella infection, though under certain circumstances it can last several months in infants. A chronic course is also possible in rare cases.

According to the Infection Protection Act, any suspicion or case of acute infectious gastroenteritis may have to be reported under certain circumstances. The discovery of salmonellae always has to be reported. There are numerous legal regulations to combat salmonellae in the EU (e.g. bovine salmonellae regulation, chicken salmonellae regulation, various regulations under feedstuff and food laws, etc). The focus of food analysis and clinical salmonellae diagnostics lies in the cultivation of the pathogen from food (including carcasses) or from stools and scat samples and in the serological identification or exclusion of salmonellae in suspicious cases by means of omni- or polyvalent salmonellae diagnostic serums. This kind of diagnosis can normally only be made approx. 1-2 days after receipt of the samples by the laboratory. A further 2-3 days are needed for a safe diagnosis of a salmonellae infection. Suspicious individual clones are characterised biochemically by means of coloured rows and further characterised serologically. The serological differentiation takes place with antiserums against O- and H-antigens in an agglutination test according to the Kaufmann-White scheme. This means that 3-5 days are needed for a definite identification of a salmonellae infection. This prolonged period until a diagnosis poses a big problem, particularly with food-induced salmonelloses, since further persons could be infected in the meantime. This is why it is important to localise the source of infections and to prevent further infections in the event of suspected salmonellae. The earliest possible diagnosis is of key importance so that this can take place quickly.

The serological identification of the antibody is primarily used in veterinary medicine and the food industry in the form of ELISA systems. Identification takes place using meat press juice, blood or swab/scratch sponge samples, for example. The disadvantage of this method is that a series of pathogenic salmonellae serovars cannot be reliably identified.

In human medicine, on the other hand, the Widal-agglutination test is used as a supplement to the bacteriological identification of pathogens for salmonellae infections. The disadvantage of this method, on the one hand, is that not all infections lead to the formation of antibodies against O-antigens and that antibodies against H-antigens can persist for years. Since titres against O-antigens often fall within a few weeks to below a detection limit, it is impossible to safely differentiate between an acute and an older infection.

During the in-vitro cultivation of salmonellae, these excrete a whole series of proteins in the culture medium. One of these proteins is the so-called SipC protein (Salmonella Invasions Protein), whose nucleotide and amino acid sequence is available in databases.

The SipC protein is one of the effector proteins that allow the invasion of salmonellae in the host cells. This invasion is enabled through the polymerisation and condensation of actin filaments. This procedure is referred to as ‘bundling’.

The bacteria are absorbed through macropinocytosis. This is preceded by an intensive exchange of signals between the pathogen and host cell. The host cell's membrane ripples, caused by eukaryotic growth factors. The rippling of the membrane produces membrane-bound vacuoles in which the salmonellae are absorbed. Under normal conditions the membrane rippling and associated bacterial invasion is prevented by microfilament inhibitors such as Cytochalasin D. This fact highlights the necessity of actin polymerisation for the bacterial invasion. It could be shown that the SipC protein alone, without the effect of other components of the host cell, is absorbed by this and leads to the ‘bundling’ of the actin filaments.

The use of PCR primers and FRET hybridisation probes against the SipC gene is described to prove salmonellae. The use of antibodies against SipC for an immunological identification was not taken into account. HAYWARD et al. (1999) and WEIHRAUCH et al. (2002) describe the SipC identification in an immunoblot using a polyclonal antibody. The disadvantage of the anti-serums used lies in their cross-reactivity. This is why they were unsuitable for a specific identification of salmonellae.

The goal of the invention is to develop a reliable test to identify salmonellae. It is based on the task of designing a method that allows the earliest possible identification and covers all important serovars.

The invention is realised in accordance with claims 1-10. The basis of the invention is the use of the SipC protein to identify a salmonellae infection. This protein is excreted during the metabolic processes of salmonellae. The crucial point of the invention is the surprising discovery that there are 5 highly antigenic regions in the SipC protein. The invention is based on the fact that these regions can be identified with antibodies and/or corresponding nucleic acid sequences. WO/2007/016912 describes the SipC protein as a highly preserved protein and points out that the amino acid sequences of the SipC protein in all salmonella serovars display only very minor differences and form the proteins of the type HI secretion system at a very early point in time. Various monoclonal antibodies against the protein form the basis for the identification of the SipC developed on this basis. This identification system permits an answer to the question as to whether an active salmonellae infection is present in the organism.

When considering the structural analysis of the amino acid sequence by means of 3 different methods, it was surprisingly discovered that there are 5 highly antigenic regions in the protein. The following amino acid sequences were identified:
1. The protein 363-378 with the sequence VASTASDEARESSRKS (SEQ ID NO 1)
2. The protein 15-30 with the sequence NNHSVENSSQTASQSV (SEQ ID NO 2)
3. The protein 343-357 with the sequence GQYAATQERSEQQIS (SEQ ID NO 3)
4. The protein 276-289 with the sequence LGIKDSNKQISPEH (SEQ ID NO 4)
5. The protein 246-260 with the sequence LNMKKTGTDATKNLN (SEQ ID NO 5)

The peptides are arranged as follows in the overall protein:

```
(SEQ ID NO 6)
                10 20 30 40 50 60
MLISNVGINP AAYLNNHSVE NSSQTASQSV SAKDILNSIG ISSSKVSDLG LSPTLSAPAP
                70 80 90 100 110 120
GVLTQTPGTI TSFLKASIQN TDMNQDLNAL ANNVTTKANE VVQTQLREQQ AEVGKFFDIS
              130 140 150 160 170 180
GMSSSAVALL AAANTLMLTL NQADSKLSGK LSLVSFDAAK TTASSMMREG MNALSGSISQ
              190 200 210 220 230 240
SALQLGITGV GAKLEYKGLQ NERGALKHNA AKIDKLTTES HSIKNVLNGQ NSVKLGAEGV
              250 260 270 280 290 300
DSLKSLNMKK TGTDATKNLN DATLKSNAGT SATESLGIKD SNKQISPEHQ AILSKRLESV
              310 320 330 340 350 360
ESDIRLEQNT MDMTRIDARK MQMTGDLIMK NSVTVGGIAG ASGQYAATQE RSEQQISQVN
              370 380 390 400
NRVASTASDE ARESSRKSTS LIQEMLKTME SINQSKASAL AAIAGNIRA
```

Although the peptides alone trigger an antibody induction, it has proven expedient in accordance with the invention to bind these peptides to normal carrier substances such as hemocyanin. Test animals such as rabbits, guinea pigs, goats, chickens or fish are immunised with peptides in a known manner to produce the polyclonal anti-peptide antibodies in accordance with the invention. In order to produce monoclonal antibodies, the peptides are used in a known manner for the induction of specific B-cells which generate hybridoma cells after fusing with myeloma cells, which are cultivated in accordance with known cloning methods and then secrete the specific monoclonal antibodies. It could be proven that the mono- or polyclonal antibodies in accordance with the invention react highly specifically with the SipC protein.

The SipC protein consists of a hydrophobic middle part (amino acids 121-199) and two hydrophilic parts. The N-terminal domains (amino acids 1-120 and the C-terminal domain (amino acids 200-409). Since the hydrophilic parts of the protein are soluble in high concentrations under physiological conditions, these and the sequence of the overall protein were expressed in comparison to the sequences of the identified immunogenic peptides in E. coli (strain BL21, vector pET28a).

```
(SEQ ID NO 6)
                10 20 30 40 50 60
MLISNVGINP AAYLNNHSVE NSSQTASQSV SAKDILNSIG ISSSKVSDLG LSPTLSAPAP
                70 80 90 100 110 120
GVLTQTPGTI TSFLKASIQN TDMNQDLNAL ANNVTTKANE VVQTQLREQQ AEVGKFFDIS
              130 140 150 160 170 180
GMSSSAVALL AAANTLMLTL NQADSKLSGK LSLVSFDAAK TTASSMMREG MNALSGSISQ
              190 200 210 220 230 240
SALQLGITGV GAKLEYKGLQ NERGALKHNA AKIDKLTTES HSIKNVLNGQ NSVKLGAEGV
              250 260 270 280 290 300
DSLKSLNMKK TGTDATKNLN DATLKSNAGT SATESLGIKD SNKQISPEHQ AILSKRLESV
              310 320 330 340 350 360
ESDIRLEQNT MDMTRIDARK MQMTGDLIMK NSVTVGGIAG ASGQYAATQE RSEQQISQVN
              370 380 390 400
NRVASTASDE ARESSRKSTS LIQEMLKTME SINQSKASAL AAIAGNIRA
```

Nucleic Acid Sequence:

(SEQ ID NO 7)
atg tta att agt aat gtg gga ata aat ccc gcc gct tat tta [symbol] [symbol]
agc gct aaa gat
att ctg aat agt att ggt att agc agc agt aaa gtc agt gac ctg ggg ttg
agt cct aca ctg agc gcg cct gcg cca ggg gta tta acg caa acc ccc gga
acg atc acg tcc ttt tta aaa gcc agt att caa aat acc gac atg aat cag
gat ttg aat gct ctg gca aat aat gtc acg act aaa gcg aat gag gtt gtg
caa acc cag tta cgc gag cag cag gca gaa gtc gga aag ttt ttt gat att
agc gga atg tct tcc agt gcc gtt gcg ctg ttg gct gcc gcg aat acg tta
atg ctg acg ttg aac cag gct gat agc aaa ctg tct ggt aag ttg tca tta
gtc agt ttt gat gca gct aaa acg acg gca agc tcc atg atg cgc gaa ggg
atg aat gcg ttg tcc ggt agt att tcc cag agc gcg ctt cag ttg ggg atc
act ggc gtg ggc gcc aaa ctg gaa tat aag ggg ctg cag aat gaa aga ggc
gcg ctt aaa cat aat gcc gcg aag atc gat aaa ctg acc act gaa agc cac
agt att aaa aac gtg ctg aac ggg cag aat agc gtc aaa ctc ggt gct gaa
ggc gtc gat tct ctg aaa tcg [symbol] [symbol]
gat gcg acg ctt aaa tct aat gcc gga acc agc gcc
acg gaa agt [symbol]
cag gct att ctg tcg aaa cgt ctt gag tct gtc gaa tcc gat att cgt ctt
gag cag aat acc atg gat atg acc cga atc gat gcg cgc aag atg cag atg
acg ggc gat ctg att atg aag aac tcg gtc acg gtc ggt ggt att gca ggg
gcg tcc [symbol]
cag gtg aat aac cgg [symbol] [symbol]
acc agc ctg att cag gaa atg ctg aaa aca atg gag agc
att aac cag tcg aaa gca tcc gca ctc gct gct atc gca ggc aat att cgc
gct taa

The details of the method in accordance with the invention will be explained further in the following. The identification of a salmonellae infection/contamination takes place in excretions of patients/animals, carcasses, eggs and foods/animal feeds, whereby the SipC of all known salmonellae serovars is identified.

This determination is carried out by means of immunochemical systems using mono- and polyclonal antibodies that are directed against the following peptide sequences:

(SEQ ID NO 1)
NH₂-V-A-S-T-A-S-D-E-A-R-E-S-S-R-K-S-COOH,
(SEQ ID NO 2)
NH₂-N-N-H-S-V-E-N-S-S-Q-T-A-S-Q-S-V-COOH,
(SEQ ID NO 3)
NH₂-G-Q-Y-A-A-T-Q-E-R-S-E-Q-Q-I-S-COOH,
(SEQ ID NO 4)
NH₂-L-G-I-K-D-S-N-K-Q-I-S-P-E-H-COOH
and
(SEQ ID NO 5)
NH₂-L-N-M-K-K-T-G-T-D-A-T-K-N-L-N-COOH

and/or with the nucleic acid sequences corresponding to the named sequences. The antibodies used in accordance with the invention are produced by means of antigens that represent the complete SipC or sub-sections of this, whereby the amino acid sequence of the complete SipC is not used.

The aforementioned synthetically produced peptides, which induce antibodies after the immunisation of animals that detect SipC and/or its hydrophilic and hydrophobic sub-sections are, also used as antigens.

The antibodies produced can be used individually or in a combination in immunochemical identification systems.

The method to obtain mono- and/or polyclonal antibodies that react specifically with SipC and are induced through common immunisation methods, is characterised by the fact that the aforementioned peptides

(SEQ ID NO 1)
NH₂-V-A-S-T-A-S-D-E-A-R-E-S-S-R-K-S-COOH,
(SEQ ID NO 2)
NH₂-N-N-H-S-V-E-N-S-S-Q-T-A-S-Q-S-V-COOH,
(SEQ ID NO 3)
NH₂-G-Q-Y-A-A-T-Q-E-R-S-E-Q-Q-I-S-COOH,
(SEQ ID NO 4)
NH₂-L-G-I-K-D-S-N-K-Q-I-S-P-E-H-COOH,
(SEQ ID NO 5)
NH₂-L-N-M-K-K-T-G-T-D-A-T-K-N-L-N-COOH

or immunogenic partial peptides of these, are used as antigens to immunise vertebrates, in particular small mammals and birds.

It has proven practical to couple the free peptides to suitable carrier substances, preferably hemocyanin or albumin, before immunisation.

Polyclonal antibodies can be produced using chickens, for example.

The objects of the invention are also the polyclonal and monoclonal antibodies used, that are produced in the manner described.

All cleaning and detection systems for SipC that contain at least one antibody against the aforementioned peptides in accordance with the invention are covered by the patent application.

The antibodies are used in immunological test kits with one or more antibodies for the diagnosis/identification of salmonellae infections/contaminations from stools and/or different matrices.

Such test kits can also contain 2 different antibodies (Sandwich-ELISA).

The method in accordance with the invention to produce a test system to identify/determine salmonellae infections/contaminations from stools samples and/or different matrices, comprises the steps:
a. Chemical synthesis of the identified peptide sequences or
b. Digestion of SipC through proteolytic/chemical fission and use of the fission products to immunise test animals with subsequent extraction of antibodies from these animals or lymphatic node/spleen/blood cells or other parts of the body and fusing of these cells with myeloma cells,
c. Extraction and purification of the antibodies produced in the animals or an egg laid by these, and isolation of monoclonal antibodies from cell culture fluid,
d. Bonding of a combination of the purified antibodies to a suitable carrier.

The fission of the proteins in the culture supernatant is carried out by means of cyanogen bromide or one or more proteases. A combination of trypsin and pepsin is preferably used as proteases. If the antibodies are produced by immunisation, the antibodies are purified from an egg laid by the chicken. Further cleaning of the antibodies can be carried out in a protein A-column or through other affinity and/or gel- or ion-chromatography methods or through fractionated precipitation. The purified antibodies can be bonded to solid carriers either chemically or by absorption.

The carrier is preferably designed as a particle, membrane or plate. It consists of nitrocellulose, cellulose acetate or PTFE membranes or the cavity of a well plate or as a flat plate or spherical particle.

In accordance with the invention, the diagnosis/identification method for salmonellae infections/contaminations consists of the steps
a. Production of a test system,
b. Incubation of the carrier with stools/stools concentrations/scat/scat concentrations/concentrations-precultivations of other substrates,
c. Identification of the proteins from the test substrates named in b) bonded to the carrier with the aid of detection antibodies.

The other substrates are pigs and parts of pigs and other mammals/birds (meat, blood, organs) environmental samples (scat, swab samples, scratch sponge samples, boot/sock samples, stable dust) food and animal feed. With this method both the detection antibodies and the purified antibodies can be produced as secondary antibodies.

In other cases, cross-reactive antibodies and/or a combination of antibodies of different origins are used.

The method is characterised by the fact that an immunochemical detection method is used, whereby this can be an ELISA, a strip or a spot assay. The detection antibodies are preferably marked, whereby biotin, peroxidase, gold or fluorescent dyes can be used. Peroxidase-marked streptavidin and/or peroxidase substrate can be used wherever practical. TMB is preferably used as a peroxidase substrate. The reaction of the peroxidase-substrate is proven by an optically visible product.

### EMBODIMENT 1

### Production of Specific Anti-Peptide-Antibodies that are Directed Against Defined Sections of the SipC Protein

Peptides with the amino acid sequence NH₂-V-A-S-T-A-S-D-E-A-R-E-S-S-R-K-S-COOH (SEQ ID NO 1), NH₂-N-N-H-S-V-E-N-S-S-Q-T-A-S-Q-S-V-COOH (SEQ ID NO 2), NH₂-G-Q-Y-A-A-T-Q-E-R-S-E-Q-Q-I-S-COON (SEQ ID NO 3), NH₂-L-G-I-K-D-S-N-K-Q-I-S-P-E-H-COOH (SEQ ID NO 4) and NH₂-L-N-M-K-K-T-G-T-D-A-T-K-N-L-N-COOH (SEQ ID NO 5) are synthesised by means of solid phase synthesis according to Merrifield. The peptides are coupled to limpet hemocyanin (KLH) by known methods (1 mg peptide/mg KLH). 300 μg each of this conjugate are used to immunise rabbits or chickens, with the addition of Freund's adjuvant. The animals are bled after being immunised 3 times. After the serum has been collected the specificity of the antiserums is tested in an ELISA. Free peptide is hereby absorbed on the surface of the cavities of well plates. Following incubation of the cavities with the antiserums these are washed thoroughly. The antigen-antibody reaction is detected in a common way using anti-rabbit and/or anti-chicken POD conjugate and TMB as a substrate. Each antiserum only reacts with the homologous peptide.

### EMBODIMENT 2

### Proof of the Specificity of the Antibodies in Accordance with the Invention

The SipC specificity can be proven in a western blot. To this end, roughly or highly purified SipC from culture supernatants of salmonellae ssp. are separated from accompanying contaminations by means of polyacrylamide gel-electrophoresis according to their relative molar mass. The protein zones from the gel are transferred to nitrocellulose with the aid of a ‘semi-dry-blotting’ apparatus. Following saturation of the free binding sites of the membrane with resuspended dry skimmed milk, the membranes are incubated with the 1:500 diluted anti-peptide antiserums. Following intensive washing of the membranes to remove all unspecific bonded antibodies, the membranes are incubated with phosphatase-marked anti-species-antibodies. The specifically bonded secondary antibodies that remain on the membrane after washing are made visible after the substrate is added. It could hereby be shown that only SipC is detected in the samples that are used.

### EMBODIMENT 3

### Determination of SipC in Culture Supernatants of Salmonellae Spp. Using the Antibodies in Accordance with the Invention in an ELISA

The SipC in culture supernatants of salmonellae ssp. is determined in a solid phase enzyme immunoassay based on the sandwich technology. A polyclonal antibody directed against epitopes of the SipC id dissolved in a carbonate/bicarbonate buffer mixture, pH 9.6, and placed in the wells of a well plate. Following incubation at 4° C. for 12 h, the free antibodies are removed by washing with PBS. The remaining free binding sites of the carrier material are blocked by a PBS buffer containing bovine serum albumin and Tween 20. Blocking takes place at room temperature for 90 min. After washing, the culture supernatants dissolved in PBS are pipetted into the wells. The 60-minute incubation at room temperature is concluded by washing. A second SipC specific polyclonal antibody that is conjugated with biotin is added to the SipC bonded to the first antibody.

Following a 30 min incubation and washing, the biotin-marked antibody is detected with peroxidase-marked streptavidin. The unbonded streptavidin is removed by the final washing process. TMB is then added as a substrate for the peroxidase and the colour reaction stopped by adding HCl after a defined time. The change in the optical density is measured. The intensity of the colour reaction is proportionate to the SipC concentration in the sample.

### EMBODIMENT 4

### Determination of the SipC in Stools/Scat Using the Antibodies in Accordance with the Invention

The SipC in stools/scat is determined by initially enriching the salmonellae for 4 to 8 h in peptone water. The SipC is then determined with a solid phase ELISA based on the sandwich technology. Individual or a corresponding mixture of several of the antibodies in accordance with the invention are hereby dissolved in a carbonate/bicarbonate buffer mixture, pH 9.6, and placed into the wells of a well plate. Following incubation at 4° C., the free antibodies are removed by washing with PBS. The remaining free binding sites of the carrier material are blocked by a PBS buffer containing bovine serum albumin and Tween 20. Blocking takes place at room temperature for 90 min. After washing, the stools samples dissolved in PBS are pipetted into the wells. The 60-minute incubation at room temperature is concluded by washing. Individual or a corresponding mixture of several antibodies in accordance with the invention that have been conjugated with biotin are used as detection antibodies. Following a 30 min incubation and washing, the biotin-marked antibody is detected with peroxidase-marked streptavidin. The unbonded streptavidin is removed by the final washing process. The peroxidase concentration is then determined with TMB as a substrate. After adding HCl to end the enzyme reaction, the change in the optical density is measured. The intensity of the colour reaction is proportionate to the SipC concentration in the sample.

### EMBODIMENT 5

### Determination of the SipC from Different Test Materials Such as

pigs and parts of pigs and other mammals/birds (meat, blood, organs)
environmental samples (scat, swab samples, scratch sponge samples, boot/sock samples, stable dust)
food and animal feed
   using the antibodies in accordance with the invention. Is determined by initially enriching the salmonellae spp. A defined amount of the test material is incubated in buffered peptone water or other enrichment media for 4-8 h at 37° C. to induce the secretion. Without any further selective enrichment and isolation, the supernatant is centrifuged and the SipC contained therein is determined with a solid phase ELISA based on the sandwich technology. Individual or a corresponding mixture of several of the antibodies in accordance with the invention are hereby dissolved in a carbonate/bicarbonate buffer mixture, pH 9.6, and placed into the wells of a well plate. Following incubation at 4° C., the free antibodies are removed by washing with PBS. The remaining free binding sites of the carrier material are blocked by a PBS buffer containing bovine serum albumin and Tween 20. Blocking takes place at room temperature for 90 min. After washing the samples diluted in PBS are pipetted into the wells. Following the 60-minute incubation at room temperature the reaction is concluded by washing. Individual or a corresponding mixture of several antibodies in accordance with the invention that have been conjugated with biotin are used as detection antibodies. Following a 30 min incubation and washing, the biotin-marked antibody is detected with peroxidase-marked streptavidin. The unbonded streptavidin is removed by the final washing process. The peroxidase concentration is then determined with TMB as a substrate. After adding HCl to end the enzyme reaction, the change in the optical density is measured. The intensity of the colour reaction is proportionate to the SipC concentration in the sample.

### EMBODIMENT 6

### Determination of the SipC from Different Test Materials Such as

pigs and parts of pigs and other mammals/birds (meat, blood, organs)
environmental samples (scat, swab samples, scratch sponge samples, boot/sock samples, stable dust)
food and animal feed
stools/scat
   in a strip quick test (direct assay)

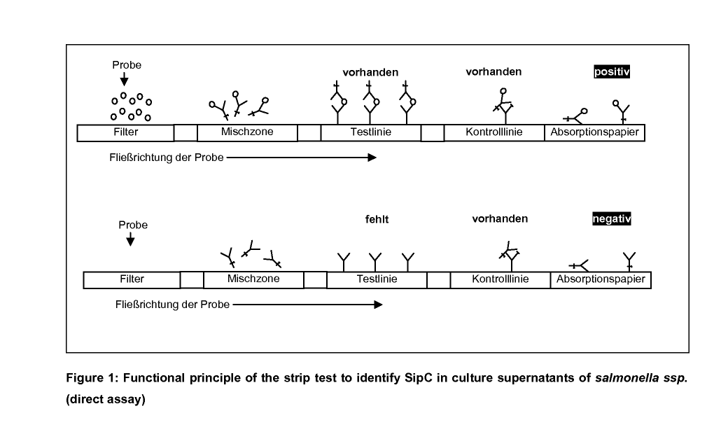

Testing Process: see FIG. 1

The SipC in culture supernatants of salmonellae ssp. is determined in a qualitative immuno-chromatographic strip test. The test is based on a specific reaction of gold-conjugated, anti-SipC-antibodies with free SipC in the sample. The test device consists of a plastic base with supported nitrocellulose membrane (Sigma Aldrich). Anti-SipC-antibodies and purified anti-species-antibodies are immobilised on two lines (test line and control line). Gold particles were bonded to the purified antibodies directed against epitopes of the SipC protein (40 nm, British Biocell International, Cardiff, GB). The conjugated antibodies were placed on a conjugate pad (Arista Biologicals, Allentown, Pa., USA). This conjugate pad overlaps with the nitrocellulose membrane. The sample is applied to the sample field. If the sample contains SipC, this bonds to the gold-conjugated antibodies. After adding sample buffer, the sample and antibodies move along the nitrocellulose membrane through capillary forces. The anti-SipC-antibodies that are resuspended by adding buffer move in the direction of the test line and control line. If SipC is bonded by the gold-marked antibodies and the protein bonds with another immunogenic determinant to the immobilised antibodies on the test line, a red line appears at this point. A red band appears on the control line as soon as the antibodies that have migrated with the sample buffer are bonded to the control line by the anti-species-antibodies. If there is no SipC in the sample the gold-marked anti-SipC-antibodies are not immobilised by the SipC protein to the antibodies on the test line and a red band only appears on the control line. The colour reaction on both lines is completed after around 10-15 minutes.

### EMBODIMENT 7

### Determination of SipC in Cultures of Salmonellae ssp. By Means of Fluorescence In-Situ Hybridisation (FISH) Using Marked Probes Against Sections of the Nucleic Acid Sequence of the SipC

The method is based on the hybridisation of oligonucleotides that are marked with a fluorochrome (e.g. CY3) to their complementary sequences on the target molecule (DNA/RNA). The oligonucleotides usually consist of 15-25 nucleotides, have a balanced ratio of A/T to G/C, have a melting point between 50° C. and 70° C., do not contain complementary regions and bear G/C-bases at their ends. The nucleic acid probes can bond to extracted nucleic acids or be used for ‘whole cell preparation’. The cells have to be permeabilized for the latter method. For Gram-negative bacteria, this is normally done by fixing the probes with (para-) formaldehyde. The hybridisation of the fluorochrome-marked probes to the target sequence depends on the accessibility of the target-DNA, the characteristics of the probe (length, melting temperature, A/T:C/G ratio) and the hybridisation conditions (buffer, temperature, incubation time). Since the DNA is normally a double strand this has to be separated beforehand. This is normally done by shifting the pH-value or increasing the temperature. During heat denaturation, the melting temperature is lowered by adding formaldehyde. The denaturation can thus be achieved at temperatures of around 70° C. The so-called stringency (bond strength at a certain temperature) of the probe to the target sequence depends on the salt, formaldehyde and sample concentration. At a constant hybridisation temperature, an increasing formaldehyde concentration and falling salt concentration leads to higher stringency. Non-bonded oligonucleotides are flushed out to the cells in a washing stage. This allows a differentiation between cells that have not been specifically bonded by the probe and the target cells. Cells that have not been bonded by the probe display no FISH fluorescence signal, but can be marked by DAPI-counterstaining. During the washing step, the temperature is the same as the incubation temperature. Only the salt concentration of the washing buffer is lower than that of the hybridisation buffer. This increases the stringency of the samples.

The following probes were successfully tested by way of example:

(SEQ ID NO 8)
5′-GGATTGCGAAGCTGTCTGTGAACTATTCTC-3′
(SEQ ID NO 9)
5′-CGTCGCATCGGTACC-3′
(SEQ ID NO 10)
5′-GAGATTTGTTTATTACTG-3′
(SEQ ID NO 11)
5′-GAACGTTCCTGAGTAGCGGC-3′
(SEQ ID NO 12)
5′-CACGGGCTTCGTCCGATGCGGTGCTGGC-3′

The probes are marked at the 5′-end with the fluorescent dye Cy3.

Following pre-cultivation of the samples under test, these are fixed with formaldehyde

(end concentration 2%) and the bacteria cells are thus permeabilized. After a 30-minute incubation at 4° C. the die batches are heated to 75° C. to denaturate the double-strand DNA. The probes are diluted in hybridisation buffer and added to the batches. Hybridisation takes place at a temperature of 45±1° C. After a 3 h incubation the batches are centrifuged off at 5000 g⁻¹ and the pellet then absorbed in an ethanol/PBS-mixture (ratio 1+1). This washing step is repeated twice to flush the free probes out of the cells. The resuspended samples are placed on a specimen slide and dried at a temperature of 45±1° C. After drying, the specimen slides are dehydrated for 2 min each in 50%, 80% and 96% ethanol and then dried in the air. After drying, the specimen slides can be analysed using a fluorescence microscope.

### EMBODIMENT 8

### Determination of SipC in Cultures of Salmonellae ssp. By Means of Nucleic Acid Amplification Techniques (PCR) Using Specific Primers for the Nucleic Acid Sequence of the SipC

PCR is generally a method to duplicate (amplify) a defined part of a DNA-strand in vitro. PCR is one of the safest detection methods for bacteria (and other organisms) on the DNA-level, but requires established information on the type and class of bacteria on account of its selectivity. The primer should be chosen to amplify a DNA-fragment that is specific for the type of organism to be identified. Primer pairs that are each complementary to a strand of the DNA sequence of the SipC protein were chosen to detect salmonellae.

The following pairs of primers were successfully tested by way of example:

| | | | Product |
|---|---|---|---|
| | | | length |
| Direction | 5′-Pos | Primer sequence (5′->3′) | (bp) |
| Forward | 25 | ATCCCGCCGCTTATTTAAATAATC | 1186 |
| | | ATTCTG (SEQ ID NO 13) | |
| Reverse | 1210 | CGATAGCAGCGAGTGCGG | |
| | | (SEQ ID NO 14) | |
| Forward | 25 | ATCCCGCCGCTTATTTAAATAATC | 1176 |
| | | ATTCTG (SEQ ID NO 15) | |
| Reverse | 1200 | GAGTGCGGATGCTTTCGACTGG | |
| | | (SEQ ID NO 16) | |
| Forward | 62 | TAGTTCACAGACAGCTTCGCAATC | 1117 |
| | | C (SEQ ID NO 17) | |
| Reverse | 1178 | TTAATGCTCTCCATTGTTTTCAGC | |
| | | ATTTCC (SEQ ID NO 18) | |
| Forward | 68 | ACAGACAGCTTCGCAATCCGTTAG | 1108 |
| | | (SEQ ID NO 19) | |
| Reverse | 1175 | ATGCTCTCCATTGTTTTCAGCATT | |
| | | TCCTG (SEQ ID NO 20) | |
| Forward | 68 | ACAGACAGCTTCGCAATCCGTTAG | 1152 |
| | | (SEQ ID NO 21) | |
| Reverse | 1219 | TATTGCCTGCGATAGCAGCGAG | |
| | | (SEQ ID NO 22) | |

The invention provides a method that allows a fast identification of salmonellae infections. Whereas the known methods need 3-5 days for a safe diagnosis, the method in accordance with the invention provides a result after approx. 10 hours.

A further advantage lies in the universal applicability; all important serovars are reliably identified.

1-10. (canceled)

11. Diagnostic method to detect a salmonellae infection/contamination, characterised in that the SipC of all known salmonellae serovars excreted by the salmonellae can be identified in excretions of patients/animals, carcasses, eggs and food/animal feed, whereby the determination is carried out by means of immunochemical systems using mono- and polyclonal antibodies that are directed against the following peptide sequences:
NH₂-V-A-S-T-A-S-D-E-A-R-E-S-S-R-K-S-COOH (SEQ ID NO 1), NH₂-N-N-H-S-V-E-N-S-S-Q-T-A-S-Q-S-V-COOH (SEQ ID NO 2), NH₂-G-Q-Y-A-A-T-Q-E-R-S-E-Q-Q-I-S-COOH (SEQ ID NO 3), NH₂-L-G-I-K-D-S-N-K-Q-I-S-P-E-H-COON (SEQ ID NO 4) and NH₂-L-N-M-K-K-T-G-T-D-A-T-K-N-L-N-COOH (SEQ ID NO 5) so that the SipC of all known salmonellae serovars can be identified.

12. Method in accordance with claim 11, wherein the antibodies used have been produced by means of antigens that represent the complete SiP-C of their sub-sections, whereby the antigens are either
obtained naturally or
produced synthetically

13. Method according to claim 12, further comprising the step of coupling the antibodies to a carrier substance, the antibodies such as hemocyanin of guanine, are used for the immunisation of animals.

14. Method according to claim 13, wherein the carrier substance is hemocyanin of guanine.

15. Method according to claim 13, wherein the animals are vertebrates.

16. Polyclonal or monoclonal antibodies produced in accordance with the method of claim 12.

17. Polyclonal antibodies in accordance with claim 16, wherein the antibodies are produced using chickens.

18. Immunological test kits in accordance with claim 11, comprising two different antibodies (Sandwich-ELISA).
